(19) United States
(12) Patent Application Publication
(10) Pub. No.: US 2010/0106097 A1
(43) Pub. Date: Apr. 29, 2010

(54) DRUG ACCEPTABILITY INDICATOR

(75) Inventor: Ahmed Elmouelhi, Maple Grove, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/568,974

(22) Filed: Sep. 29, 2009

Related U.S. Application Data
(60) Provisional application No. 61/109,387, filed on Oct. 29, 2008.

(51) Int. Cl. A61M 5/31

(52) U.S. Cl. 604/189

(57) ABSTRACT

The invention relates generally to an indicator for a syringe. More particularly, the present invention relates to a method and apparatus for testing the compatibility of a fluid before injecting the fluid into a medical device. An indicator may be attached to an interior of the reservoir body wherein the indicator strip contacts the fluid medication loaded into the reservoir body and visually indicates the presence of a selected parameter. The indicator may be an indicator strip infused with an chemical indicator that changes color when in contact with a selected target material.

RELATED APPLICATION

This application claims the benefit of the filing date of a provisional U.S. application Ser. No. 61/109,387, filed Oct. 29, 2008.

### FIELD

This invention relates generally to an indicator for a syringe. More particularly, the present invention relates to a method and apparatus for testing the compatibility of a fluid before injecting the fluid into a medical device.

### BACKGROUND

Infusion devices may be used to deliver an infusion media (e.g. a medication such as insulin) to a patient. Such devices may be designed to be implanted into a patient's body to deliver predetermined dosages of the infusion media to a particular location within the patient's body, e.g. in the venous system, the spinal column, or within the peritoneal cavity.

Infusion devices, and other types of pumps and medical devices, are normally refilled at periodic intervals when the reservoir of the drug or medication is running low. Often, clinicians who refill the medical device are not the same person who actually creates or mixes the drug to be injected into the medical device. If the clinician is not intimately familiar with the additive, preservative, or other material being injected into the medical device it is possible that material could be injected into the medical device that is harmful to the continued operation of the device. It is therefore desirable to have simplified testing methods and indicators to insure that a solution containing a drug or other medicant is safe for injection into the pump or other medical device.

One previous example of a system and method for determining the material in a syringe is described in U.S. patent application Ser. No. 11/564,348. The '348 application describes a syringe for use in spectroscopy to identify the drugs within the syringe. The syringe includes an optical window such that the syringe can be loaded into a spectroscopic analyzer to determine a spectroscopic fingerprint of the liquid contained in the syringe. The spectroscopic fingerprint can then be compared to spectroscopic data of known drugs to determine the drugs present. Such an operation may be relatively time consuming and complicated to run, requiring specific equipment and expertise.

There is therefore a need for a simple indicator system that checks for selected materials or impurities and provides a simple indication to a user regarding compatibility of the fluid for injection into a device.

### BRIEF SUMMARY OF THE INVENTION

According to an aspect of the invention, an indicator is placed into a syringe in which a fluid, for example, a medication, is to be injected into a medical device. In one embodiment the medical device is an implanted medical device such as a drug pump. The indicator will give instant or near instant feedback regarding various compatibility parameters between the medical device and the fluid to be injected therein.

### BRIEF DESCRIPTION OF THE DRAWINGS

Embodiments of the present invention will hereinafter be described in conjunction with the following drawings wherein like reference numerals denote like elements throughout, and

FIG. 1 is a perspective view of an implantable infusion device in accordance with one embodiment of the present invention.

FIG. 2 is a representative view of an infusion device implanted into a body of a patient in accordance with one embodiment of the present invention

FIGS. 3A-3D are schematic representations of infusion systems implanted in various locations in a patient.

FIG. 4 is a perspective view of one embodiment of the present invention therein.

FIG. 5 is a perspective view of an alternative embodiment of the present invention therein.

### DETAILED DESCRIPTION OF THE INVENTION

The present invention is an indicator mounted in a syringe or other delivery device that tests a fluid therein and the fluid's compatibility to a medical device into which the fluid is to be injected. In one embodiment the syringe is being utilized to inject infusion media, such as baclofen, into an infusion device, such as a drug pump, implanted into a patient. An indicator located inside the syringe is exposed to the infusion media and may change color depending on whether a selected target material is detected. In various embodiments the indicator can be used to detect specific additives, preservatives, or other materials known to be harmful to the operation of the drug pump. One particular material that may be particularly harmful to an infusion device 10 as previously described is sodium metabisulfate. The indicator may be referred to as an indicator, test strip, indicator strip, or the like.

In further embodiments, the indicator may more generally test the characteristics of the infusion media, such as for acidity or basicity. In still further embodiments the indicator may be designed to simultaneously respond to a range of different materials such that different portions of the indicator change color in response to different materials. In various embodiments the indicator may be an indicator strip, a test strip, or other media that can be exposed or immersed to a fluid medicant for testing purposes. In one embodiment the indicator strip may be provided separately such that a small amount of the infusion media to be injected can first be applied to the indicator strip to determine compatibility. One advantage to the present invention is that it may give real time or short term information to the user regarding specific characteristics of the fluid to be injected. In similar applications the indicator strip may be placed inside other delivery apparatuses besides a syringe, such as in a catheter delivering infusion media to a patient.

The following detailed description is of the presently contemplated mode of implementing the invention. This description is not to be taken in a limiting sense, but is merely for the purpose of illustrating the general principles of embodiments of the invention. Furthermore, there is no intention to be bound by any theory presented in the preceding background of the invention or the following detailed description of the invention. The scope of the invention is defined by the appended claims.

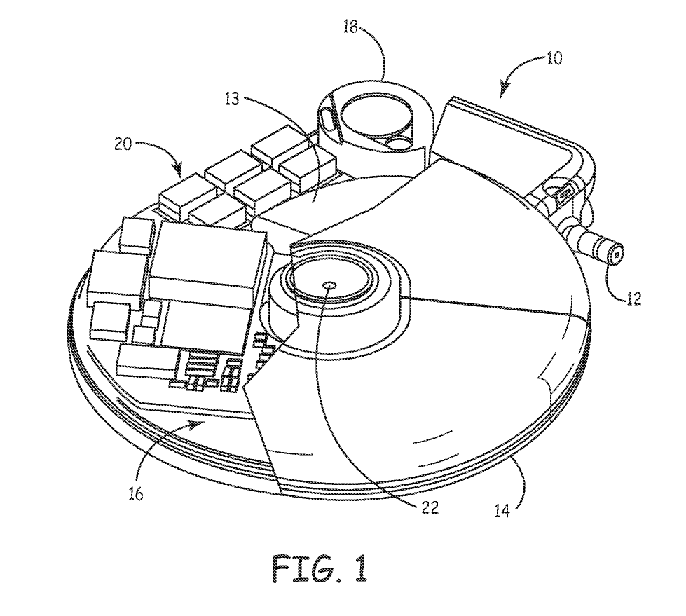

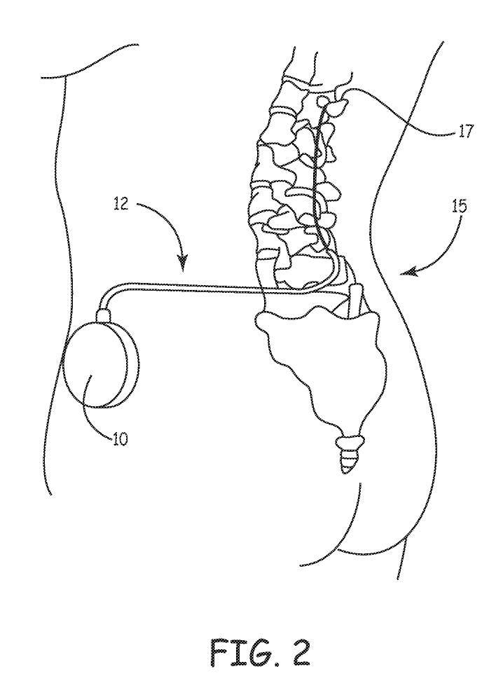

FIGS. 1-2 show an implantable infusion device 10. The illustrated device 10 is configured to be surgically implanted into a patient, for example, in the abdominal region, between the skin and the abdominal wall. The device 10 may be alternately referred to as infusion device 10, medical device 10, or as device 10. A catheter 12 connected to the infusion device 10 may deliver infusion medium to the patient, for example, but not limited to, by feeding infusion medium to a particular location in the venous system, within the spinal column, or in the peritoneal cavity of the patient. Other embodiments of the infusion device 10 may be implemented as external infusion devices that connect to patients through suitable catheter devices or the like. Yet further embodiments of the infusion device 10 may be used in other contexts, e.g., for delivery of a medium into other suitable environments. For purposes of simplifying the present disclosure, the term “patient” is used herein to refer to any environment in which an implantable device is implanted or to which an external device is connected, whether or not it is carried out for medical purposes. Also, the term “infusion medium” is used herein to refer to any suitable medium delivered by such a device 10.

In further embodiments, the present invention indicator may be utilized in connection with medical devices that are not an infusion device 10. However, for simplicity, the present description will utilize an infusion device 10 as an example.

The device 10 may include a generally disc-shaped housing 14. While a generally circular disc-shaped embodiment is illustrated in FIG. 1, it will be understood that further embodiments of the infusion device 10 may employ a housing of other shapes, including, but not limited to, oval, oblong, rectangular, or other curved or polygonal shapes. Generally, the housing 14 is made of a biocompatible material and most often has a relatively small diameter and thickness to reduce patient trauma during implant surgery and after implantation.

The housing 14 includes a reservoir 16 for holding a volume of infusion medium, such as, but not limited to, a liquid medication to be administered to the patient. Housing 14 may also contain a drive mechanism 18 (e.g. a pump), a power source 13, and control electronics 20. Inlet structure 22 may provide a closeable and sealable fluid flow path to the reservoir 16 in the reservoir portion of the housing such as an elastomeric septum. The inlet structure 22 may include a port for receiving a needle through which fluid may be transferred to the reservoir 16, for example, to fill or re-fill the reservoir 16 of the device with the infusion media or a rinsing fluid. The infusion device 10 may also include an outlet structure 22 for attaching the catheter 12.

In particular embodiments, the inlet structure 22 may be configured to re-seal after a fill or re-fill operation, and to allow multiple re-fill and re-seal operations. One example of an inlet structure is described in U.S. Pat. No. 6,652,510, titled “Implantable Infusion Device and Reservoir for Same,” which is incorporated by reference herein in its entirety and for everything it teaches and discloses. However, further embodiments may employ other suitable inlet structures, including, but not limited to, those described in U.S. Pat. Nos. 5,514,103 and 5,176,644, each to Srisathapat et al.; U.S. Pat. No. 5,167,633 to Mann et al.; U.S. Pat. No. 4,697,622 to Swift et al.; and U.S. Pat. No. 4,573,994 to Fischell et al., also incorporated by reference. Representative examples of reservoir housing portions and reservoirs which may be employed in embodiments of the invention are described in the above referred to U.S. Pat. No. 6,652,510, and further embodiments may employ other suitable reservoir configurations, including, but not limited to, those described in the above referred to U.S. Pat. Nos. 5,514,103; 5,176,644; 5,167,633; 4,697,622; and 4,573,994.

FIG. 2 illustrates an example placement of one embodiment of an implantable infusion device 10 that is implanted within a patient's body 15. The exemplary infusion device 10 depicted is an implantable medical device 10 that may include at least one catheter 12. Such infusion devices 10 may be used for a wide variety of therapies including treatment of pain, spasticity, and other medical conditions.

The medical device 10 and catheter 12 are typically implanted by a clinician (e.g., surgeon) within the body 15 during a surgical procedure. The medical device 10 is, in the illustrated embodiment, operable to infuse a fluid from an enclosed reservoir into the body 15 through the catheter 12. The present invention also contemplates embodiments wherein the catheter is implanted with a proximal end outside the body 15 so that it may attach to an external infusion device, the remainder of this description is, for the sake of brevity, directed to implantable infusion devices 10 that are entirely implanted in the body 15 of the patient.

Before implantation of the medical device 10, the catheter 12 may be positioned such that the fluid delivered to the patient through the catheter 12 reaches a selected internal delivery location 17 within the body 15 of the patient. As depicted, the infusion device 10 is implanted such that the delivery site 17 is located within the intrathecal space of the spinal canal. The infusion device 10 of the present invention may be used to deliver fluid to any other selected internal delivery location.

Catheter 12 may preferably disgorge fluid at its distal end. In other embodiments, catheter 12 may have a delivery region that is not at the distal end of the catheter 12, e.g., a hole or valve positioned somewhere before reaching the distal end of the catheter 12.

A proximal end of the catheter 12 may be tunneled through the tissue to the infusion device 10 implant location and coupled to a catheter port 12a of the device 10. If implanted, the medical device 10 is typically positioned subcutaneously, e.g., from 1 centimeter (0.4 inches) to 2.5 centimeters (1 inch) below the skin, where there is sufficient tissue for supporting the medical device 10 with sutures or the like.

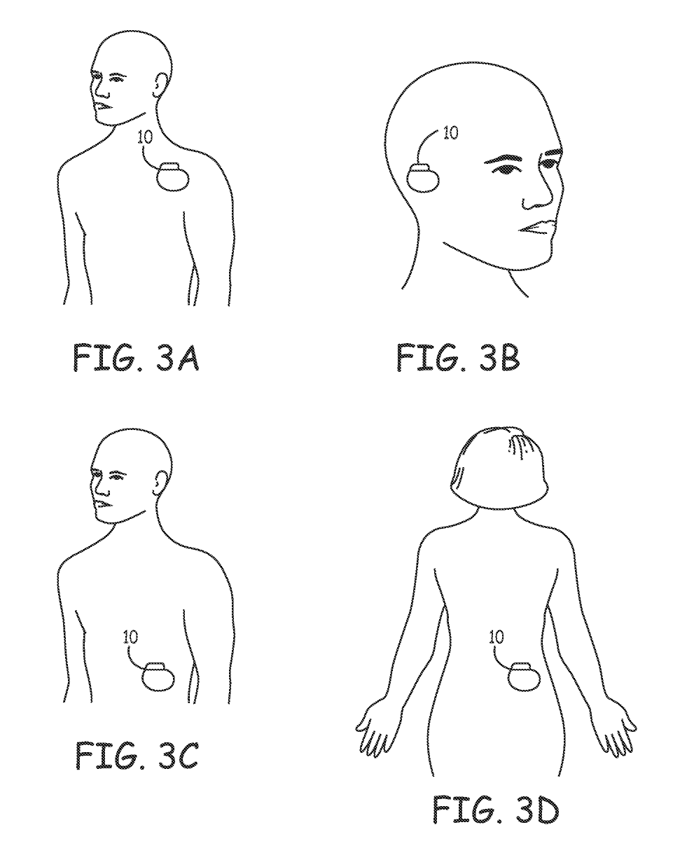

Referring to FIGS. 3A through 3D, alternative locations for implanting infusion device 10 are shown. As depicted in FIG. 3A, device 10 may be implanted in the pectoral region of a patient. Alternatively, infusion device 10 may be implanted behind the patient's ear (FIG. 3B), in the patient's abdomen (FIG. 3C), or in the patient's lower back or buttocks (FIG. 3D). Of course, infusion device 10 may be placed in any medically acceptable location in patient 15.

Once the device 10 is implanted into the body, the device 10 must be filled with the selected infusion media for treating the patient 15. A typical device for filling the device 10 is a syringe with a needle tip.

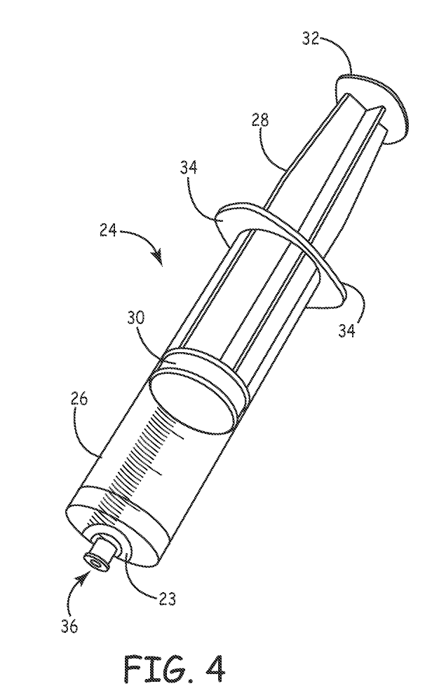

As illustrated in FIG. 4, the present invention is illustrated as an indicator strip 23 placed in a syringe 24. The syringe 24 may be a standard syringe that includes a reservoir body 26, a plunger 28, and an elastomeric plunger head 30 located on a distal end of the plunger 28. The plunger 28 may also include a plunger flange 32 adapted to be pressed by a user's thumb while the user's fingers are engaged with reservoir flanges 34 located on a proximal end of the reservoir body 26. As is well known by those of skill in the art, the elastomeric plunger head 30 should be of a size and shape to correspond to the reservoir body 26 such that when the plunger flange 32 is utilized to press down the plunger 28, material contained in the syringe 24 is expelled from an outlet 36 located on a distal end of the reservoir body 26. In the present embodiment, wherein the syringe 24 is utilized to fill the reservoir 16 of the implantable drug pump 10, the outlet 36 may be fitted with a needle that is used to pierce the patient 15 and the pump inlet 22. The reservoir body 26 may also include volume indications 27 on the side.

The indicator strip 23 may be placed on an interior distal end 38 of the syringe 24 reservoir body 26. The interior end 38 of the reservoir body 26 may be transparent so that a clear view of the indicator strip 23 is available. In further embodiments, the interior end 26 of the syringe 24 may be translucent or colored in such a manner that the visual indication provided by the indicator strip 23 (as further described below) can be observed by the clinician.

The indicator strip 23 may be a circular strip placed around outlet 26 in a substantially circular pattern. The indicator strip 23 may be adhered to the interior end 26 with an adhesive or by other methods known to those in the art. As may be appreciated, the adhesive used to adhere the indicator strip 23 to the end of the syringe 26 should be generally inert such that any material that may be placed in the syringe 24 for injection into the drug pump 10 would not be affected by the adhesive and would also not absorb, dissolve, or otherwise take up any of the adhesive such that the adhesive is injected into the drug pump 10.

The ring shaped indicator strip 23 may be made of paper or other material onto which a test material as described below can be loaded, infused, or impregnated with an indicator. Various types of suitable media may be known to those in the art, such as sterile filter paper or chromatography paper that are infused with an indicator.

In general, the indicator is an organic substance that indicates by a change in its color the presence, absence, or concentration of some other substance. In the present embodiment, the color change is visible to the naked eye of the user and would preferably occur over a short time duration after exposure.

One suitable indicator strip 23 material may be litmus paper. Litmus paper may be infused with various types of dyes that have a known color change when contacting materials of a certain pH. In one embodiment the strip may be a universal pH test strip. Other pH indicators that may be used may include phenolphthalein, thymol blue, phenol red, or the like. Placement of such an indicator strip 23 in the syringe 24 reservoir body 26 may indicate to the user that the liquid medicant is not at a suitable pH for injection into the body or into the infusion device 10.

In further embodiments the indicator strip 23 may indicate contact with materials of a certain classification or of a specific type. One material that may be particularly harmful to an infusion device 10 as previously described is sodium metabisulfate. In one example, therefore, the indicator strip 23 may be formed of a sulfite detection paper that indicates the presence of a sulfite, sulfurous acid, or sulfur dioxide. One example of a sulfite testing paper is available from Sigma-Aldrich at www.sigma-aldrich.com. In further embodiments other indicator strips 23 may be placed in the syringe 24 to indicate the presence of other types of materials using a variety of indicators, including materials relevant to the medical device area or in other areas as well, such as for injection of materials directly into human or animal patients.

As may be appreciated, the size of the indicator strip 23 may affect the volume of fluid that can be contained in the reservoir body 26. In most cases the volume taken up by the indicator strip 23 may be de minimus But, in some embodiments the volume indications 27 on the reservoir body 26 may take this volume into account.

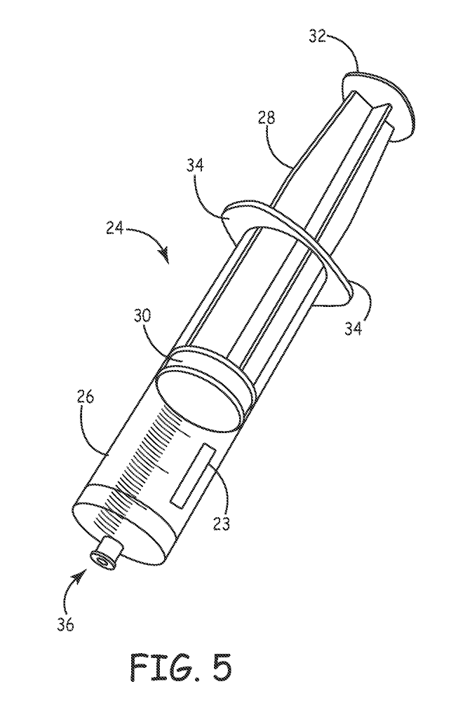

In another embodiment shown in FIG. 5, the indicator strip 23 may be placed along a length of the reservoir body 26. In such an embodiment the indicator strip 23 should be of a thickness whereby the elastomeric plunger head 30 still provides a good seal with the reservoir body 26 such that actuation of the plunger 28 ejects the fluid through the outlet 36 and the fluid does not leak past the plunger head 30. Modifications to the shape of one or more of the plunger head 30 and reservoir body 26 may be utilized to effectuate an acceptable seal while accommodating the indicator strip 23 along the interior long axis of the reservoir body 26.

In further embodiments where the indicator strip 23 may be designed to detect the presence or absence of more than one material. In such a case visual markings on the syringe may be utilized to indicate what sections of the indicator strip 23 may change color to represent various parameters of the fluid to be injected.

In further embodiments the indicator strip 23 may be placed in a separate chamber in the syringe 24. In such an embodiment the syringe may include a small chamber in the reservoir body 26 or next to the reservoir body 26 wherein the liquid medicant flows into the chamber and makes contact with the indicator strip 23. As may be appreciated by those of skill in the art, such a separate chamber could be designed in a number of different ways, including internal to the reservoir body 26 (wherein the plunger shape may need to be altered to accommodate the chamber) or external to the reservoir body 26 and connected to the reservoir body 26 in such a manner that provides fluid flow from the reservoir body 26 to the chamber, either through a one way valve, capillary action, or some other manner Utilization of a separate chamber from the syringe body 26 may partially or substantially isolate the fluid medicant that contacts the indicator strip 23 from injection into the infusion device 10. Such segregation of the fluid medicant from the material injected into the infusion device may minimize concerns relating to drug dilution, contamination, or breakdown caused by the indicator strip 23.

Still further embodiments the indicator strip may be separate from the syringe 24 such that a small amount of the fluid to be injected into the drug pump 10 can be placed on the strip for testing.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing exemplary embodiments of the invention, it being understood that various changes may be made in the function and arrangement of elements described in an exemplary embodiment without departing from the scope of the invention as set forth in the appended claims and their legal equivalents.

What is claimed is:

1. A syringe for delivery of a fluid medication comprising
a reservoir body;
a plunger operably attached to a proximal end of the reservoir body, the plunger including a plunger head on the distal end of the plunger;
an outlet located on a distal end of the reservoir body;
an indicator fixedly attached to an interior of the reservoir body wherein the indicator contacts the fluid medication loaded into the reservoir body and visually indicates the presence of a selected parameter.

2. The syringe of claim 1 wherein the indicator is fixedly attached to the distal end of the reservoir body.

3. The syringe of claim 1 wherein the indicator is a litmus paper.

4. The syringe of claim 1 wherein the indicator is a sulfite detection paper.

5. The syringe of claim 1 wherein the selected parameter is pH.

6. An apparatus for delivering a fluid, the apparatus comprising:
a housing;
an inlet in the housing for receiving the fluid;
an outlet in the housing for discharging the fluid;
a piston channel within the housing through which the fluid flows from the inlet to the outlet;
an actuator positioned within the housing and moveable between a first position and a second position, the actuator defining a piston chamber for storing fluid received through the inlet when the actuator is in the retracted position, the actuator driving the fluid stored in the piston chamber toward the outlet when the actuator transitions from the retracted position to the forward position and;
an indicator fixedly attached to the piston channel for indicating a parameter of the fluid.

7. The syringe of claim 6 wherein the indicator is fixedly attached to the distal end of the reservoir body.

8. The syringe of claim 6 wherein the indicator is a litmus paper.

9. The syringe of claim 6 wherein the indicator is a sulfite detection paper.

10. The syringe of claim 6 wherein the selected parameter is pH.

11. A device for delivery of a fluid medication comprising
a reservoir body;
a plunger operably attached to a proximal end of the reservoir body, the plunger including a plunger head on the distal end of the plunger;
an outlet located on a distal end of the reservoir body;
a chamber fluidly connected to the reservoir body wherein placement of a fluid in the reservoir body results in some amount of the fluid entering the chamber;
an indicator fixedly attached to an interior of the chamber wherein the indicator contacts the fluid medication loaded into the reservoir body and visually indicates the presence of a selected parameter.
